(19) United States
(12) Patent Application Publication
(10) Pub. No.: US 2017/0273695 A1
(43) Pub. Date: Sep. 28, 2017

(54) MEDICAL APPARATUS INCORPORATING A SYSTEM FOR PERFORMING REMOTE ISCHEMIC CONDITIONING

(71) Applicant: CellAegis Devices Inc., Toronto (CA)

(72) Inventors: Rocky Eugene Ganske, Acton (CA); Lahav Gil, Toronto (CA); Igal Roytblat, Richmond Hill (CA)

(73) Assignee: CellAegis Devices Inc., Toronto, ON (CA)

(21) Appl. No.: 15/505,638

(22) PCT Filed: Aug. 20, 2015
(86) PCT No.: PCT/IB2015/001856

§ 371 Date: Feb. 22, 2017

Related U.S. Application Data
(60) Provisional application No. 62/040,669, filed on Aug. 22, 2014.

(51) Int. Cl. A61B 17/135; A61N 1/39; A61B 5/021

(52) U.S. Cl. CPC A61B 17/1355; A61B 5/021; A61N 1/39; A61B 6/032

(57) ABSTRACT

A remote ischemic conditioning system may be integrated with other medical devices or apparatus so that cycles of remote ischemic conditioning may be performed on a patient before, during and/or after performance of another medical procedure. In one embodiment, a remote ischemic conditioning system may be integrated with a surgery table. In another embodiment, a remote ischemic conditioning system may be integrated with an automated external defibrillator, or an automated external defibrillator/monitor. Integration of the remote ischemic conditioning system with the surgical table or the automated external defibrillator allows the remote ischemic conditioning system to be powered by the power source for the table or the automated external defibrillator, and/or to be controlled by a control panel disposed on the table or the automated external defibrillator. Also, data from the controller for the remote ischemic conditioning system may be received and stored in the controller for the table or the automated external defibrillator. This data may be displayed on a monitor for the table or defibrillator and/or transmittal to a hospital or heart center.

### FIELD

This invention relates generally to systems for performing remote ischemic conditioning (RIC), and more particularly to the integration thereof into other medical apparatus and devices used to perform a medical procedure or treatment other than RIC.

### BACKGROUND OF INVENTION

Ischemic diseases are significant causes of mortality in industrialized countries. It is well established that tissue damage may result from ischemia (insufficient blood flow to a tissue) followed by reperfusion (reflow of blood to the tissue). Ischemia and reperfusion cause disturbance of micro-circulation with ensuing tissue damage and organ dysfunction. Organs such as the kidney, heart, liver, pancreas, lung, brain and intestine are known to sustain damage following ischemia and reperfusion.

In ischemic conditioning (IC), a tissue or organ or region of a subject's body is deliberately subjected to brief ischemic periods, followed by a brief reperfusion episode. IC has been found to render the tissue, organ or region resistant to injury during subsequent ischemic episodes. The phenomenon of ischemic conditioning has been demonstrated in most mammalian tissues. IC is now recognized as one of the most potent innate protective mechanisms against ischemia-reperfusion injury.

Remote ischemic conditioning (RIC) refers to the deliberate induction of a transient ischemic period in a subject at a region remote from at least some of the tissue to be protected followed by a reperfusion period. The ischemic period may involve complete cessation of blood flow (blood flow occlusion). Such ischemic periods may be induced by applying supra-systolic pressures on a region of the body, such as a limb. Alternatively, ischemic periods may also be induced by applying less than systolic pressure. Often, RIC includes inducing transient ischemia in a subject's limb to protect organs remote from the limb, such as the myocardium. Myocardial protection has been demonstrated by a variety of remote stimuli, including renal ischemia, liver ischemia, mesenteric artery ischemia, and skeletal muscle limb ischemia.

RIC may be performed prior to, during or following an ischemic injury or other injury which benefits from RIC. RIC has shown benefit in reducing or preventing damage resulting from myocardial infarction and trauma. The use of remote ischemic conditioning to improve outcome after a myocardial infarction is described in US 2011/0240043, the contents of which are incorporated herein by reference in their entirety. Similarly, the use of remote ischemic conditioning to treat traumatic injury is described in US 2011/0251635, the contents of which are incorporated herein by reference in their entirety. In addition, remote ischemic conditioning has been demonstrated to be useful in the treatment and prevention of restenosis, as described in US 2011/0190807, the contents of which are incorporated herein by reference in their entirety.

### SUMMARY

In one embodiment, there is provided a method of configuring performance of remote ischemic conditioning (RIC) on a patient that is being treated using a medical apparatus that is adapted to perform RIC and at least one second medical procedure. The method comprises operating at least one processor of the medical apparatus to carry out acts of monitoring one or more biological characteristics of the patient during performance of one or more of the at least one second medical procedure on the patient using the medical apparatus, evaluating the one or more biological characteristics to identify a manner in which to perform RIC on the patient using the medical apparatus, and configuring the medical apparatus to perform RIC on the patient in the manner identified as a result of the evaluating.

In another embodiment, there is provided at least one non-transitory computer-readable storage medium having encoded thereon executable instructions that, when executed by at least one processor of a medical apparatus adapted to perform remote ischemic conditioning (RIC) and at least one second medical procedure, cause the at least one processor to carry out a method of configuring the medical apparatus to perform RIC on a patient. The method comprises monitoring one or more biological characteristics of the patient during performance of one or more of the at least one second medical procedure on the patient using the medical apparatus, evaluating the one or more biological characteristics to identify a manner in which to perform RIC on the patient using the medical apparatus, and configuring the medical apparatus to perform RIC on the patient in the manner identified as a result of the evaluating.

In a further embodiment, there is provided a method of operating a medical apparatus that is adapted to perform remote ischemic conditioning (RIC) and at least one second medical procedure on the patient. The medical apparatus is disposed within an emergency vehicle transporting the patient to a medical facility. The method comprises generating, during performance of RIC on the patient using the medical apparatus, first data regarding the performance of the RIC on the patient, generating, during performance of the at least one second medical procedure on the patient, second data regarding the performance of the at least one second medical procedure on the patient, triggering wireless transmission of at least one message comprising the first data and the second data to the medical facility.

In another embodiment, there is provided an apparatus comprising an inflatable cuff and a controller to control inflation of the inflatable cuff in accordance with a protocol. The controller comprises at least one processor and at least one storage medium having encoded thereon executable instructions that, when executed by the at least one processor, cause the at least one processor to carry out a method. The method comprises, in response to user input requesting operation of the apparatus as a blood pressure monitor, controlling inflation of the inflatable cuff in accordance with a first protocol associated with blood pressure monitoring, and in response to user input requesting operation of the apparatus to perform remote ischemic conditioning (RIC) on a patient, controlling inflation of the inflatable cuff in accordance with a second protocol associated with RIC and rendering the inflatable cuff inoperable once RIC has been performed using the inflatable cuff.

In a further embodiment, there is provided at least one non-transitory computer-readable storage medium having encoded thereon executable instructions that, when executed by at least one processor, cause the at least one processor to carry out a method of controlling inflation of an inflatable cuff of a medical apparatus in accordance with a protocol. The method comprises, in response to user input requesting operation of the medical apparatus as a blood pressure monitor, controlling inflation of the inflatable cuff in accordance with a first protocol associated with blood pressure monitoring and, in response to user input requesting operation of the medical apparatus to perform remote ischemic conditioning (RIC) on a patient, controlling inflation of the inflatable cuff in accordance with a second protocol associated with RIC and rendering the inflatable cuff inoperable once RIC has been performed using the inflatable cuff.

In another embodiment, there is provided an apparatus comprising one or more pressurized gas cylinders, an air pump, an inflatable cuff, a battery, at least one processor, and at least one computer-readable storage medium having encoded thereon executable instructions that, when executed by the at least one processor, cause the at least one processor to carry out a method comprising, in response to user input requesting operation of the apparatus as a blood pressure monitor, operating the air pump to inflate the inflatable cuff and sensing a blood pressure of a patient to which the inflatable cuff is attached and, in response to user input requesting performance of remote ischemic conditioning (RIC) on a patient using the apparatus, triggering inflation of the inflatable cuff using a pressurized gas cylinder of the one or more pressurized gas cylinders.

In a further embodiment, there is provided medical apparatus comprising a system for performing remote ischemic conditioning, the system comprising an inflatable cuff configured to encircle a limb of a patient and to occlude blood flow through the limb of the patient, a controller attached to the cuff and configured to perform a remote ischemic conditioning procedure on a patient, and a pump for providing gas to inflate the inflatable cuff, the pump being controlled by the controller. The medical apparatus further comprises apparatus for performing a medical procedure on a patient and a data link between the system for performing remote ischemic conditioning and the apparatus for performing a medical procedure.

In another embodiment, there is provided a table comprising apparatus for performing a medical procedure, a table surface upon which the medical procedure may be performed, and a system for performing remote ischemic conditioning on a patient before, during and/or after performance of the medical procedure. The system for performing remote ischemic conditioning comprises an inflatable cuff configured to encircle a limb of a patient, and to occlude blood flow through the limb of the patient, a controller attached to the cuff, the controller being configured to alternately inflate and deflate the cuff to alternately occlude blood flow and to allow blood to flow through the limb during a programmed cycle of remote ischemic conditioning, and a pump for inflating the cuff under the control of the controller. The table further comprises a data link between the first control panel and the controller of the remote ischemic conditioning system to allow the transmission of data between the remote ischemic conditioning system controller and the first control panel.

In a further embodiment, there is provided an automated external defibrillator comprising paddles or pads for applying a charge to a patient, a first controller for controlling operation of the paddles or pads in accordance with automated external defibrillation, a system for performing remote ischemic conditioning. The system comprises a cuff configured to encircle a limb of a patient, a pump for providing a gas to the cuff for inflation of the cuff, and a second controller for controlling the pump to inflate and deflate the cuff to perform cycles of remote ischemic conditioning to the patient, each cycle including a period of occlusion of blood flow through the limb followed by a period of reperfusion of blood flow as the cuff is deflated. The automated external defibrillator further comprises a data link between the control panel on the first controller and the second controller to provide power to the second controller and to permit data transmission between the second controller and the first controller.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. Various embodiments of the invention will now be described, by way of example, with reference to the accompanying drawings, in which:

FIG. 1 is a schematic perspective view of an assembled system for remote ischemic conditioning with a removable controller;

FIG. 2 is a schematic perspective view of the system for remote ischemic conditioning depicted in FIG. 1 with the controller removed;

FIG. 3 is a cross sectional view of the system for remote ischemic conditioning depicted in FIG. 1 taken along the line 3-3 in FIG. 1;

FIG. 4 is an exploded schematic perspective view of the cuff of the system depicted in FIG. 1;

FIG. 5 is a schematic top perspective view of the controller attachment section of the system depicted in FIG. 1;

FIG. 6 is a schematic bottom perspective view of the controller attachment section of the system depicted in FIG. 1;

FIG. 7 is a schematic bottom perspective view of the controller of the system depicted in FIG. 1;

FIG. 8 is a schematic top perspective view of the controller of the system depicted in FIG. 1;

FIG. 9 is a cross sectional view of the controller and controller attachment section while coupled to the system depicted in FIG. 1;

FIG. 9A is a detailed view of FIG. 9 corresponding to box A of FIG. 9;

FIG. 10 is a schematic perspective view of the controller of the system depicted in FIG. 1 with the cover removed;

FIG. 11 is a schematic perspective view of the controller of the system depicted in FIG. 1 with the cover and PCB removed;

FIG. 12 is a schematic perspective view of a charging cradle to be used with the controller;

FIG. 13 is a schematic perspective view of the charging cradle of FIG. 12 with an optional wall mount;

FIG. 14 is a partial, perspective view of a table system having an integrated system for remote ischemic conditioning;

FIG. 15 is a partial, perspective view of the control panel of the table system of FIG. 14;

FIG. 16 is a partial, perspective view of one embodiment of the docking station and associated remote ischemic conditioning controller of the control panel of FIG. 15;

FIG. 17 is a perspective view of another embodiment of the docking station and remote ischemic conditioning controller of the control panel of FIG. 15;

FIG. 18 is a pictorial representation of a system for remote ischemic conditioning integrated with an automated external defibrillator;

FIG. 19 is a perspective view of the defibrillator of FIG. 18 and the controller for a remote ischemic conditioning system;

FIG. 20 is a flowchart of a process that may be used in some embodiments to transmit data generated by a combined medical device to a medical facility;

FIG. 21 is a flowchart of a process that may be used in some embodiments to configure a combined medical device to perform RIC and another medical procedure;

FIG. 22 is a flowchart of a process that may be used in some embodiments to configure performance of RIC on a patient in response to conditions detected during performance of another medical procedure;

FIG. 23 is a flowchart of a process that may be used in some embodiments to regulate use of components of a combined medical device in response to operating the combined medical device to perform RIC; and

FIG. 24 is a block diagram of some components of an example of a combined medical device.

### DETAILED DESCRIPTION

The illustrative embodiments described herein are not necessarily intended to show all aspects of the invention. Aspects of the invention are not intended to be construed narrowly in view of the illustrative embodiments. It should be appreciated that the various concepts and embodiments introduced above and those discussed in greater detail below may be implemented in any one of numerous ways, as the disclosed concepts and embodiments are not limited to any particular manner of implementation. In addition, it should be understood that aspects of the invention may be used alone, or in any suitable combination, with other aspects of the invention.

In one aspect, a system for performing RIC is integrated into another medical apparatus or device used in the performance of another medical procedure, including another medical treatment. In one embodiment, a system for performing RIC is incorporated into a device for measuring blood pressure. In another embodiment, a system for performing RIC is integrated into a surgical table which may be used in conjunction with surgery, a three-dimensional imaging device, such as a computed tomography device, or a magnetic resonance imaging device. In other embodiments, the system for performing RIC may be used with a fluoroscopy system in conjunction with an angioplasty procedure, the insertion of stents into arteries, or angiography. In yet other embodiments, a system for performing RIC may be incorporated into an automated external defibrillator used either in an emergency vehicle, or an office, public or hospital setting. Other systems with which the RIC system may be integrated include an automated tourniquet device. In these embodiments, the RIC system may be useful for treating trauma, improving outcomes during or after myocardial infarction, or treating and/or preventing restenosis.

In each of these embodiments, the RIC system may be operated before, during or after a procedure performed in conjunction with the system or device into which the RIC system is integrated. For example, if used in conjunction with a magnetic resonance imaging device, or a computed tomography system, or with a fluoroscopy system, RIC may be performed on a subject before, during and/or after a procedure being performed. Where a stent is being inserted into the arteries of a subject, RIC may be performed on the subject before, during and/or after the insertion of the stent to reduce or prevent restenosis, to minimize any injury resulting from any trauma, or to improve an outcome should the subject suffer a myocardial infarction during the procedure.

In the embodiment where the RIC system is incorporated into a blood pressure measurement device, RIC may be performed prior to measurement of the blood pressure, or in conjunction with measurement of blood pressure, or even after the blood pressure has been measured. In this embodiment, the system with the RIC device incorporated into the blood pressure measuring device may be used in an emergency vehicle or in a hospital setting after a subject has arrived at the hospital. In the embodiment where the RIC system has been incorporated into an automated external defibrillator, RIC may be performed before, during and after treatment of the subject with a defibrillator to minimize any damage from trauma, or to improve an outcome from a myocardial infarction from which the subject may be suffering.

In another aspect, a system for performing RIC includes an inflatable cuff, a controller attachment section joined to the cuff, and a controller selectively removable from the controller attachment section. The controller may control the inflation and deflation of the inflatable cuff. Furthermore, the controller may include a control circuit programmed to implement an RIC protocol. In yet another aspect, the cuff may be soft, rigid, and made from thermoformable materials.

Turning now to the figures, several embodiments are described in further detail.

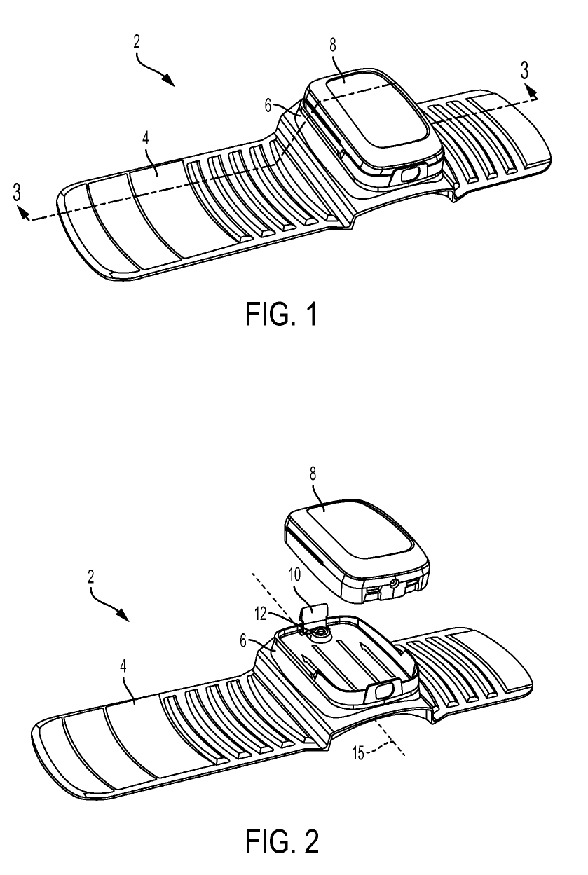

FIGS. 1 and 2 illustrate one embodiment of a system 2 for RIC. System 2 may include an inflatable cuff 4, a controller attachment section 6, and a controller 8. In some embodiments, as depicted in FIG. 2, the controller 8 is selectively removable from system 2. The controller attachment section 6 may include an interlocking retaining tab 10 adapted to provide removable attachment of the controller. The controller attachment section may also include a conduit 12 that provides, sealed, fluid communication between the controller 8 and inflatable cuff 6.

In one aspect, cuff 4 is axially rigid while being soft or non-irritating to the skin. In one embodiment, cuff 4 may include an inner layer 16, a layer 18, and a selectively inflatable bladder 20 disposed between layers 16 and 18, as depicted in FIG. 4. Cuff 4 may be adapted to encircle a limb of an individual. Axis 15 represents the approximate center of a circular configuration formed when cuff 4 is wrapped about a patient's limb. An axial direction of cuff 4 corresponds to the approximate direction of axis 15. Cuff 4 has a longitudinal direction extending down the length of cuff 4 which is substantially perpendicular to the above defined axial direction. Cuff 4 may also be intended to be a disposable item for use with removable controller 8. Inner layer 16 typically is positioned adjacent to, and often in contact with, the skin of an individual wearing system 2. Since inner layer 16 may be in contact with skin, the inner layer may be made from a soft and/or non-irritating material. The inner layer 16 may be made from a knit, woven, or felted cloth. The cloth may include either natural or synthetic materials. Possible cloths include brushed polyester, brushed nylon, and/or other suitable materials as would be apparent to one of skill in the art. Alternatively, inner layer 16 may be made from a foam. In some embodiments, inner layer 16 may be further adapted to provide moisture absorption, wicking, and/or breathability to cuff 4.

In some embodiments, cuff 4 may include two sections 22 spaced apart in a longitudinal direction and an intermediate section 24 disposed between the sections 22. Intermediate section 24 may be constructed to have a greater rigidity than sections 22. The increased rigidity of the intermediate section 24 may be created either by an inherent material property difference, a difference in the physical construction (e.g. a thicker section and/or inclusion of reinforcing features), or both. In one embodiment, the intermediate section 24 may include a substantially flat outer surface 25 for attachment to the controller attachment section 6. Intermediate section 24 may also include an inner surface 26 which is curved in the longitudinal direction of the cuff 4. The curved inner surface 26 may be constructed so as to generally conform to the curvature of a limb. In some embodiments, the size and curvature of the cuff 4 may be suited for a variety of sizes and ages of patients ranging from neonates to obese adults. The cuff 4 may also be sized for either attachment to an arm or a leg. The intermediate section 24 may be constructed from thermosetting plastics, thermoforming plastics, and/or foamed materials. Sections 22 and the intermediate section 24 may be integrally formed with one another, or they may be formed separately and subsequently joined using any appropriate method including, but not limited to, a sewn seam, ultrasonic welds, adhesives, rivets, clamping structures, and/or mechanically interlocking features. Section 22 may be formed of a foam material or any other suitably flexible yet strong material.

In one embodiment, cuff 4 may also include a plurality of reinforcing structures 28 substantially aligned in the axial direction of the cuff assembly. Reinforcing structures 28 typically may be formed in outer layer 18 of sections 22. Reinforcing structures 28 provide axial rigidity to the cuff 4. The increased axial rigidity provided by reinforcing structures 28 helps to distribute the pressure applied by cuff 4 in the axial direction to provide a substantially uniform pressure across the axial width of the cuff 4. Reinforcing structures 28 may also help to prevent kinks in cuff 4 when it is placed around the arm or leg of an individual. Reinforcing structures 28 may be spaced apart in a longitudinal direction to permit the cuff 4 to easily bend around an encircled limb while still providing increased axial rigidity. Reinforcing structures 28 may be curved or straight in shape in the axial direction. In some embodiments, the reinforcing structures 28 may be integrally formed with the foam in sections 22 such as by the application of heat and/or pressure (e.g. thermoforming) to selectively melt and/or compress portions of the foam in sections 22. The uncompressed and/or unmelted portions of foam in sections 22 form the raised reinforcing structures 28. Alternatively, reinforcing structures 28 may be separately formed and subsequently joined to sections 22.

Layer 18 may also include a cloth layer 19 applied to an exterior surface. Cloth layer 19 may be formed of a low stretch or non-stretch cloth. The low stretch or non-stretch properties may be an inherent property of the cloth selected. Alternatively, cloth layer 19 may be a made from thermoformable materials and may be laminated to the exterior surface of layer 18. The lamination process may alter the thermoformable fabric to be a low stretch or non-stretch material. In one embodiment, the cloth may be applied to and laminated with layer 18 in a flat layout prior to forming reinforcing structures 28. Reinforcing structures 28 may subsequently be thermoformed to a final desired shape. The resulting sections 22 may be soft and have low stretch or non-stretch properties. Furthermore, sections 22 may be thermoformable enabling subsequent processing steps.

Selectively inflatable bladder 20 may be disposed between inner layer 16 and layer 18. Bladder 20 may have a valve 30 arranged and adapted to provide a fluid inlet to the interior of bladder 20. Valve 30 extends through a hole 32 in the intermediate section 24 of cuff 4. Valve 30 may be placed in sealed fluid communication with a corresponding structure 33 on controller attachment section 6 which may also be in sealed fluid communication with an outlet 48 of controller 8. When connected to outlet 48 of controller 8 through structure 33 of the controller attachment section 6, valve 30 may provide pressurized gas such as air to bladder 20. In some embodiments, bladder 20 may be a component separate from layers 16 and 18. Bladder 20 may be formed such as by bonding two separate sheets of thermoplastic polyurethane together. In other embodiments, bladder 20 may be formed from air impermeable layers incorporated into layers 16 and 18 of cuff 4. Layers of bladder 20 may be bonded together in an air tight manner using any number of methods including adhesives, ultrasonic welding, beads of material around the edges, and/or other appropriate methods as would be apparent to one of skill in the art. Bladder 20 may also be formed as a unitary structure without separate layers.

Layers 16, 18, 19, and bladder 20 of cuff 4 may be held together at their edges in any suitable fashion, such as by a binding material 36 wrapped around the edge of cuff 4 and sewn to cuff 4, as shown in FIG. 4. Alternatively, cuff 4 may be held together using adhesives, rivets, ultrasonic welds, or other appropriate methods as would be apparent to one of skill in the art.

In one aspect, it may be desirable to provide a non-slip interface to prevent cuff 4 from moving on the limb of a subject, since system 2 may be worn for protracted periods of time. To provide a non-slip interface, at least one non-slip structure 34 may be disposed on the face of inner layer 16. The non-slip structure 34 may be printed, glued, sewn, applied as a bead of material using a guided tool, or by hand. The non-slip structure 34 may include, but is not limited to, one or more strips of silicone.

The cuff 4 may also include fasteners to hold the cuff on a limb of a subject and to adjust the circumferential size of the cuff 4 when in the fitted state. Such fasteners include, but are not limited to, hook and loop fasteners, latches, ratchet mechanisms, clasps, snaps, buckles, and other appropriate structures as would be apparent to one of skill in the art. For example, the fastner may be a hook and loop fastener including a plurality of adjacent unconnected hook sections 38a disposed on layer 18 or 19 and loop sections 38b disposed on inner layer 16. Hook sections 38a may extend in the axial direction of the cuff 4. The width of each hook section 38a, with respect to the longitudinal direction of the cuff, may be selected to provide a flexible cuff able to wrap around different sized limbs.

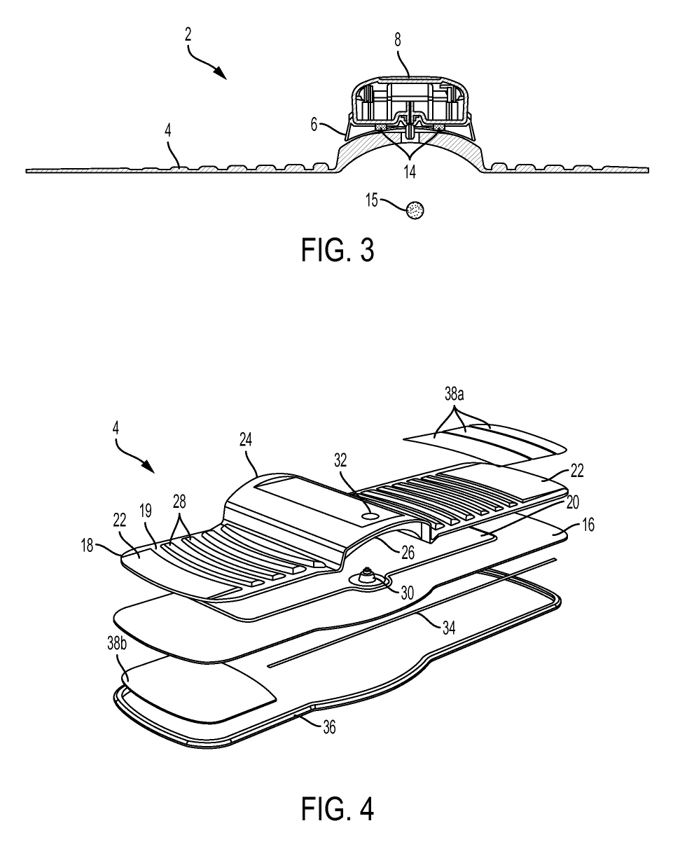

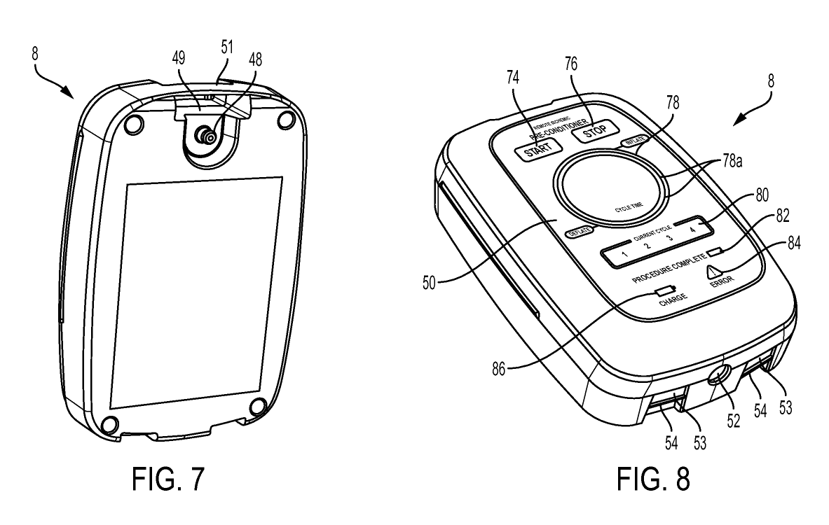

The controller attachment section 6 of FIG. 1 is shown in more detail in FIGS. 3, 5 and 6. In one embodiment, controller attachment section 6 may include an upper surface 40 for supporting controller 8 in the attached state, a lower surface 44, and an upstanding wall 42 surrounding surface 40. A raised portion 43 of upstanding wall 42 may be located adjacent to and block a power inlet 52 of controller 8 in the attached state. By blocking access to power inlet 52 in the attached state, raised portion 43 may prevent use of the device while controller 8 is connected to an external power source. The controller attachment section 6 may also include a connector, such as retaining tab 10, arranged to provide removable attachment of controller 8. In one embodiment, tab 10 is mounted at one end to surface 40 and includes a projecting edge 41 spaced from surface 40 that faces outwardly towards wall 42. Bosses 45 are disposed on wall 42 on the opposite side of section 6 from tab 10. When controller 8 is attached to attachment section 6, the upper portion of tab 10 is pushed inwardly away from wall 42 so that it passes through slot 49 that is disposed between the body of controller 8 and an outer band 51, as shown in FIG. 7. At the same time, bosses 45 extend into recesses 53 of controller 8, as shown in FIG. 8. Tab 10 has sufficient resilience that when snapped into place, this resilience creates an outward bias on tab 10 that causes edge 41 to overlie the upper edge of band 51. To release controller 8, the upper portion of tab 10 is again pushed inwardly against its bias toward controller 8 until edge 41 overlies slot 49 and is clear of band 51 at which time controller 8 may be pulled out of attachment section 6 at the end closest to tab 10.

In one embodiment, lower surface 44 and/or bottom edge 46 of controller attachment section 6 may be disposed on and substantially conform to the shape of an outer surface of cuff 4. In some embodiments, bottom surface 44 and/or bottom edge 46 of the controller attachment section 6 may be disposed on and substantially conform to the shape of outer surface 25 of intermediate section 24 of cuff 4 shown in FIG. 4. As shown in FIG. 3, the controller attachment section 6 may be joined to outer surface 25 of intermediate section 24 of inflatable cuff 4 along lower surface 44 by at least one and typically two attachment joints 14. In one embodiment, the attachment joint(s) 14 may be oriented substantially parallel to axis 15 of the cuff. The attachment joint 14 may be formed using any appropriate method including, but not limited to, a sewn seam, an ultrasonic weld, an adhesive, and/or rivets. When two or more attachment joints 14 are included, the joints 14 may be spaced apart in the longitudinal direction to allow the cuff 4 to bend and conform to the shape of different sized limbs.

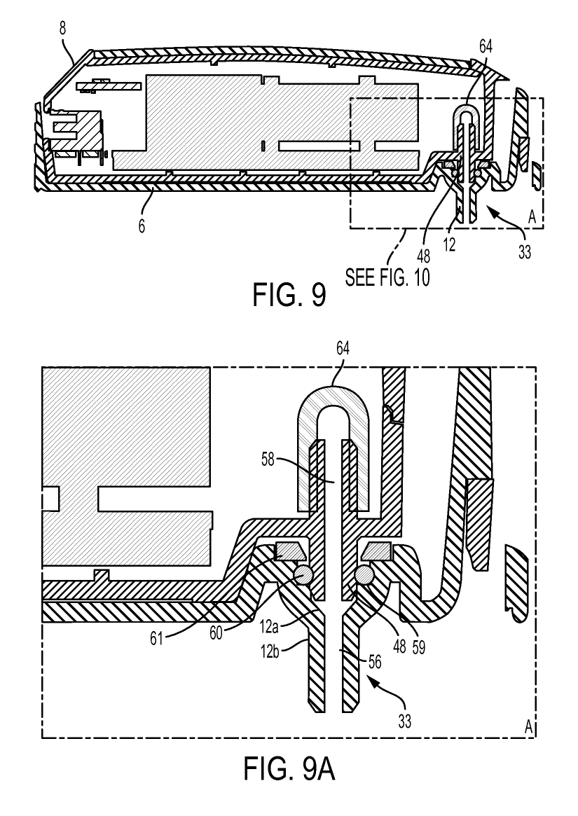

As shown in FIGS. 9 and 9A, controller attachment section 6 may provide fluid communication between the controller 8 and bladder 20 of cuff 4 via structure 33. Structure 33 may include a conduit 12 which is provided in a location spaced from retaining tab 10, when the controller 8 is in an attached state. Conduit 12 fluidly couples controller 8 to valve 30 of bladder 20. Conduit 12 may include a female section 12a that is constructed and arranged to mate with an outlet 48 of controller 8 and a male section 12b that is constructed and arranged to mate with valve 30 of bladder 20. While a male and female connection have been described, the male and female portions could be reversed or even replaced with other comparable fluid connections, such as a tube or the like. A seal, such as O-ring 60, may be disposed on a shoulder 59 located in structure 33. The O-ring 60 may create a gland seal between female section 12a and outlet 48. Alternatively, a compression seal with O-ring 60 may be used. A retaining structure 61 may be included in structure 33 to retain O-ring 60. Retaining structure 61 may be joined to structure 33 using any appropriate method including, but not limited to, press fitting, ultrasonic welding, and/or adhesives.

As shown in FIG. 8, controller 8 has a front cover 50, which may include controls and displays, and a power inlet 52. Guide structures 54 may be included in controller 8 for alignment and/or engagement with a charging mechanism

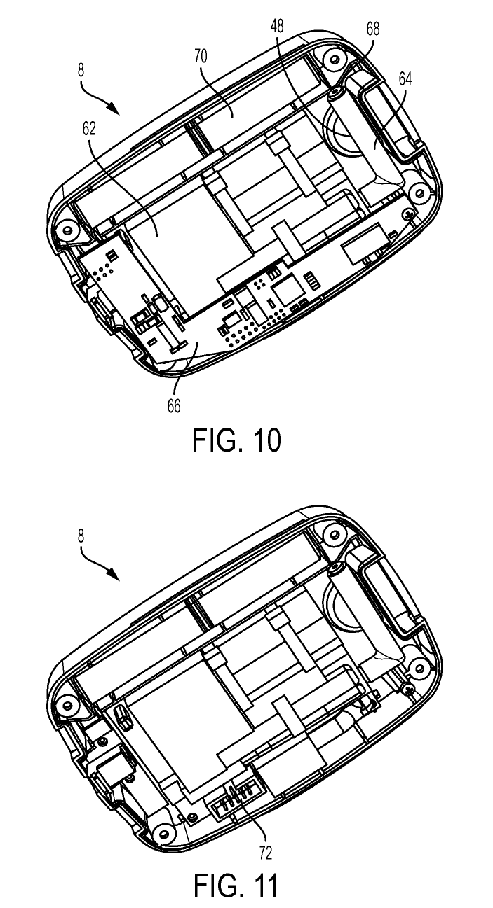

The internal components of controller 8 are best shown in FIGS. 10 and 11, where front cover 50 of controller 8 has been removed. Controller 8 may include a pump 62 in fluid communication with a manifold 64. Manifold 64 is in fluid communication with relief valve 68 and outlet 48. Controller 8 may also include a printed circuit board (PCB) 66 which may include a control circuit and memory. The controller 8 may also include a pressure sensor associated with the pressurized components of the system and the control circuit. The pressure sensor (not shown) may be incorporated into pump 62 and/or placed in pressure sensing communication with manifold 64. Furthermore, the pressure sensor may communicate with the control circuit of PCB 66. The control circuit may be programmed to implement an RIC treatment protocol. The controller may also determine blood pressure during, or as part of, an RIC treatment protocol. To provide convenient mobile usage of system 2, batteries 70 may be arranged, typically in series, to provide a higher operating voltage. Alternatively, batteries 70 may be in electrical communication with a transformer adapted to provide a higher operating voltage. In one embodiment, the operating voltage may be approximately 5 to 6 VDC. In other embodiments, the operating voltage may be approximately 12 VDC or any other appropriate voltage. As shown in FIG. 11, PCB 66 may be connected to the other controller components through plug connector 72.

The control circuit of PCB 66 may be programmed with certain error conditions which may cause the procedure to be aborted or which may cause an indication of the error to appear on a display or which can be used in other known ways. These error conditions may include, but are not limited to: the cuff is not pressurized within a predefined period, such as 20 seconds, 30 seconds, 40 seconds, 50 seconds, or one minute; there is no communication between pump 62 and PCB 66 upon start up; there is no communication between pump 62 and PCB 66 for more than a predefined period, such as two, three, four, or five seconds; multiple consecutive repumps are needed to maintain cuff pressure; pump 62 continues to run and does not respond to an abort signal after it is sent a predefined number of times, such as three, four, or five times; pressure in cuff 4 is not near zero gage pressure within a predefined period, such as 20 seconds, 30 seconds, 40 seconds, 50 seconds, or one minute after the end of an inflation cycle; pressure in cuff 4 is above a predetermined pressure such as 200, 220, 240 or 260 mmHg for longer than a predefined period, such as 5, 10, 20, or 30 seconds; and the pump 62 CPU does not wake up after a command is sent to it by the control circuit. The error condition may be cleared and/or the system may be reset such as by pressing a stop button 76 on the face of controller 8.

During usage, controller 8 may be attached to controller attachment section 6 to place controller outlet 48 into fluid communication with cuff 4. Pressurized gas may then be pumped through controller outlet 48 to inflate the cuff 4. The cuff pressure may be controlled by selectively opening valve 68 in response to a command from the control circuitry of PCB 66. In some embodiments, valve 68 may include a pressure safety relief feature that opens valve 68 in response to an over pressure event during an RIC treatment. In one embodiment, valve 68 opens when the pressure in cuff 4 exceeds 260 mmHg Valve 68 may open in response to either an error command from the control circuitry of PCB 66, or the valve 68 may include an automatically actuated mechanical system. Controller 8 may also include a slow continuous relief valve. Such a valve would continuously release gas from inflated bladder 20 at a selected rate lower than the rated flow rate of the pump 62. The slow continuous release of gas from bladder 20 could be used to deflate bladder 20 in case of a mechanism failure.

In some embodiments, the control circuit of PCB 66 may be programmable by a health professional and/or an end user according to a prescribed treatment protocol. Alternatively, the control circuit may only be programmed at the factory and may not be altered afterwards by the end user. The control circuitry may also include non-volatile memory for the logging and storage of treatment history. A health care professional may be able to access this memory to determine the treatment history of a patient and determine compliance with a prescribed treatment regime. In another embodiment, the controller may send this information via wireless, or hard wired, communication to a separate receiver for patient records, monitoring, or call center purposes. In one embodiment, controller 8 may include a start button 74 and stop button 76. In some embodiments, the start and stop buttons 74 and 76 may be incorporated into a single button. Controller 8 may also include a hard wired and/or emergency stop button and/or a quick release valve (not shown). In other embodiments, other controls may be included to allow expanded control of an RIC treatment.

In addition to controls, controller 8 may include displays related to the current cycle, the number of cycles left in a treatment, whether the treatment is completed, error signals, charge of the system, and other relevant information. In one embodiment, controller 8 may include a cycle time display 78. Cycle time display 78 may indicate the remaining portion of the inflation/deflation cycle by using illuminated indicators 78a arranged in a circular pattern corresponding to a full inflation/deflation cycle. Each indicator 78a of cycle time display 78 may correspond to a set fraction of the inflation/deflation cycle. When all of the indicators 78a of cycle time display 78 are illuminated, the inflation/deflation cycle is complete. Alternatively, the indicators 78a of cycle time display 78 may start a cycle fully illuminated and sequentially turn off as the cycle proceeds. When each indicator 78a of cycle time display 78 is dark, the particular inflation/deflation cycle is complete. While a circular display has been disclosed, cycle time display 78 could also be arranged in other linear, or non-linear, shapes corresponding to a full cycle. Controller 8 may also include a current cycle display 80, or a digital numeric display, indicating whether the current cycle is the first, second, third, or other cycle. A procedure complete indicator 82 may be illuminated with a solid color or it may blink when the RIC treatment is complete to indicate the end of the procedure. An error display 84 may indicate when an error has occurred by blinking or being fully illuminated. Alternatively, error display 84 may blink in a preset pattern or display a particular color to indicate which error has occurred. A battery charge indicator 86 may indicate the approximate charge remaining in the batteries 70, and may also signal that that the remaining charge is only sufficient for one cycle by blinking.

The above described system may be used for implementing an RIC treatment. The treatment includes placing cuff 4 on a limb of a user and attaching controller 8 to controller attachment section 6 on cuff 4. A user may then press start button 74 to initiate the treatment. Once started the control circuitry of PCB 66 monitors the pressure sensor and turns pump 62 on to inflate the cuff 4. The pressure is then increased to a desired pressure, such as a blood flow occlusion pressure. In one embodiment, the control circuitry of PCB 66 maintains the cuff pressure between preselected pressure limits such as 200 mmHg to 210 mmHg. In other embodiments, the control circuitry of PCB 66 may first determine a systolic blood pressure. After determining a systolic blood pressure, the control circuitry of PCB 66 may subsequently initiate the RIC treatment protocol with a desired pressure such as a pressure greater than the measured systolic blood pressure. Regardless of the specific pressure used, the pressure may be maintained for a selected ischemic duration. Ischemic durations may last on the order of seconds or minutes. After completing the ischemic duration, the controller may activate valve 68 to deflate cuff 4 and initiate the reperfusion duration. Reperfusion durations generally last for at least a minute, although shorter reperfusion durations may be used. After completion of the reperfusion duration another RIC cycle may be conducted. An RIC treatment may include a single cycle or multiple cycles. In one embodiment, an RIC treatment may include four cycles with ischemic durations of approximately 5 minutes, and reperfusion durations of approximately 5 minutes. At the end of the last cycle the cuff 4 may deflate within 30 seconds and the controller 8 may confirm a near zero gage pressure prior to shutting down.

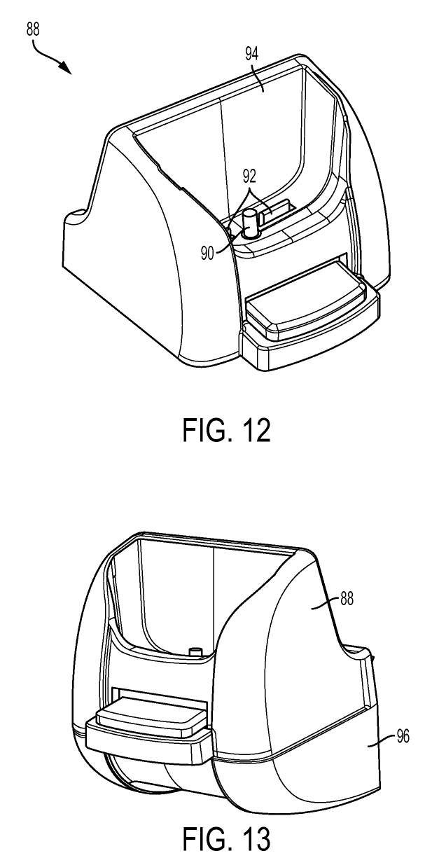

In some embodiments, controller 8 may be charged using a charging cradle 88, as shown in FIG. 12. Charging cradle 88 may include a power connector 90 and mating guide structures 92. In one embodiment, mating guide structures 92 on the charging cradle mate with guide structures 54 on the controller. Mating guide structures 92 act as alignment features. In other embodiments, mating guide structures 92 may be actuated when controller 8 is inserted into the charging cradle 88 to turn the power on and off to power connector 90. Charging cradle 88 may also include a raised area 94 to prevent insertion of the controller while controller 8 is connected to cuff 4 or a patient. In addition to the above, charging cradle 88 may optionally connect with a wall mount portion 96 as shown in FIG. 13.

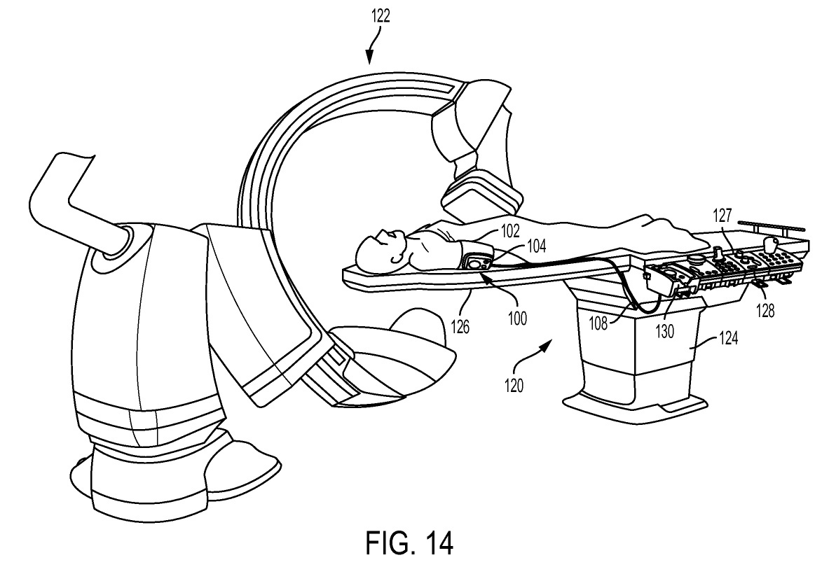

In another aspect of the invention, the RIC system of FIGS. 1-11 may be integrated with other medical apparatus to permit the performance of a remote ischemic conditioning procedure or regimen on a patient before, during or after another medical procedure that is being performed with the combination device. The other medical procedure may be a medical treatment. In one example, as shown in FIG. 14, an RIC system 100 may be used in conjunction with a table system 120 that provides support for a subject who is undergoing a procedure or treatment. In various embodiments, table system 120 may be used in conjunction with surgery, angioplasty, placement of a stent or stents, and/or angiography. Table system 120 may also be employed for surgical procedures other than the ones set forth above. Table system 120 may or may not be used in conjunction with an imaging device 122. One non-limiting example of an imaging device 122 is the Artis 1 sold by Siemens Corporation. Other examples of imaging devices are computed tomography systems, magnetic resonance imaging systems and fluoroscopy systems. Table system 120 may include a support 124, a patient support surface 126, a controller 127 and a control panel 128.

Like system 2, RIC system 100 includes an inflatable cuff 102 and a controller 104. Controller 104 may be removably attached to inflatable cuff 102 in the same manner as discussed above with respect to FIGS. 1 and 2, using a controller attachment section (not shown) which may be the same as attachment section 6. Alternatively, controller 104 may be fixedly attached to inflatable cuff 102. Inflatable cuff 102 may have substantially the same configuration and construction as discussed above with respect to inflatable cuff 4. Controller 104 may be substantially the same as controller 8, in one embodiment. RIC system 100 may be used to perform a RIC procedure as well as to measure diastolic and systolic blood pressure between cycles of RIC. In another embodiment, controller 104 may be substantially the same as controller 8, except that controller 104 does not include any batteries but does include a pump (not shown) similar to pump 62. Including the pump in controller 104 minimizes any interference with the imaging path which could be caused by pneumatic tubing.

Power to controller 104 may be provided by a cable 108 which is connected to a power source for table system 120, and which provides the necessary direct current voltage of about 5-12 volts as discussed above with respect to controller 8.

In another embodiment, cable 108 may also include a data link between controller 104 and controller 127 associated with table system 120. In this manner, the progress of any RIC procedure being performed by RIC system 100 may be controlled and monitored by the controller 127. Also, measurements of systolic or diastolic blood pressure made by RIC system 100 may be provided to controller 127 for monitoring by an attending medical practitioner or surgeon. The data link between controller 104 and controller 127 may also be wireless. In some embodiments in which the data link is wireless, controller 104 may include a power source independent of controller 127, such as batteries disposed in a same housing as controller 104 or a wire connecting controller 104 to a power source that is independent of a power source supplying controller 127. In other embodiments including a wireless data link, however, controller 104 may have a wired power connection to power source that also supplies controller 127.

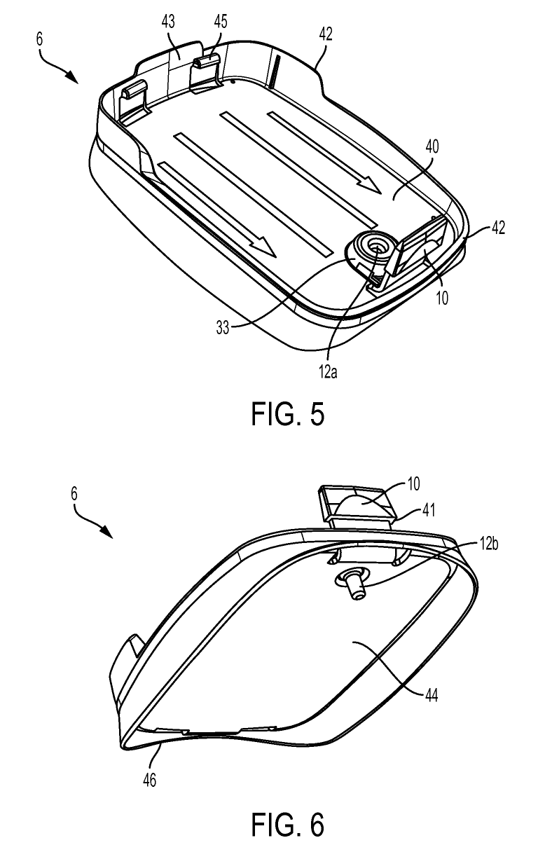

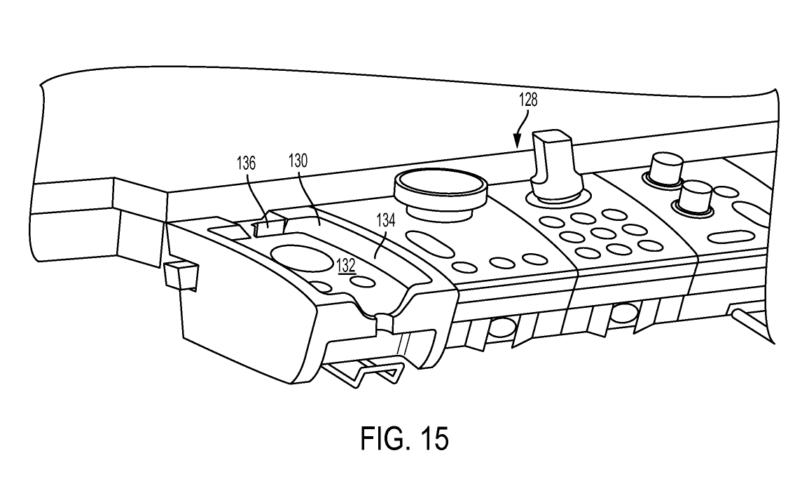

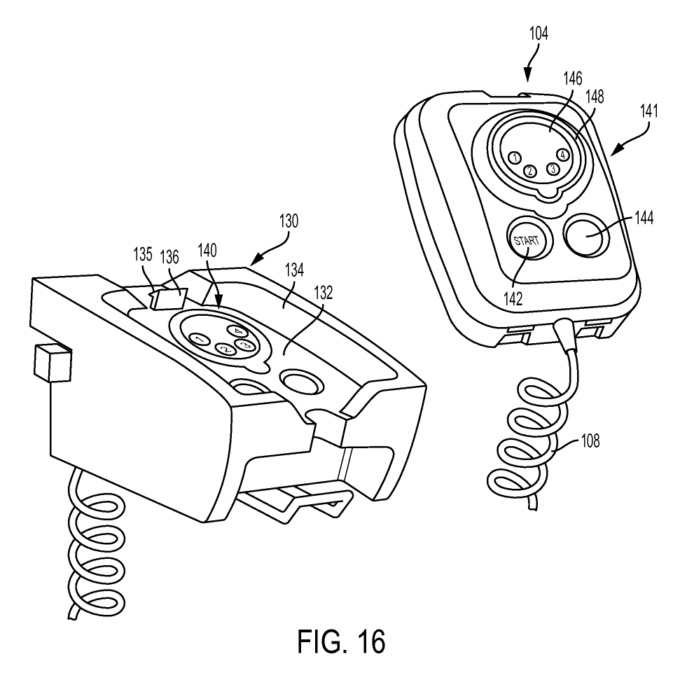

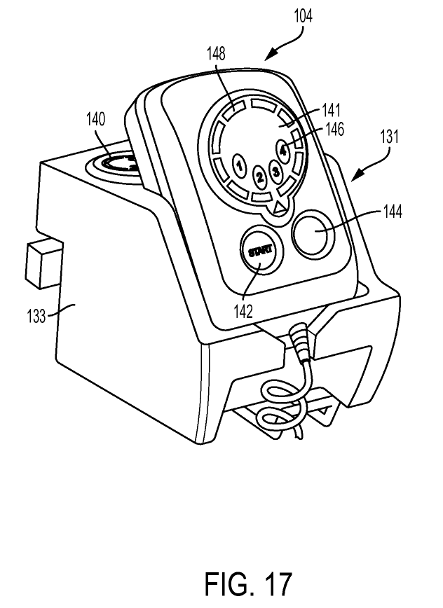

When RIC system 100 is not in use, controller 104 may be removed from inflatable cuff 102 and placed in a docking station 130 on control panel 128. Typically, docking station 130 is integrated into control panel 128 of table 120, as shown in FIG. 15. In one embodiment, as shown in FIGS. 15 and 16, docking station 130 may include an upper surface 132 for supporting controller 104, and an upstanding wall 134 which surrounds upper surface 132. A retaining tab 136 may be employed to provide removable attachment of the controller. Retaining tab 136 may be substantially identical to tab 10 as shown in FIGS. 5 and 6. Tab 136, in one embodiment, is mounted at a lower end to upper surface 132 and includes a projecting edge 135 spaced from surface 132 that faces outwardly toward wall 134. When controller 104 is attached to docking station 130, the upper portion of tab 136 is pushed inwardly away from wall 134 so that it passes through a slot, similar to slot 49 on controller 8, which is disposed between the body of controller 104 and an outer band, such as outer band 51 shown in FIG. 7. Tab 136 has sufficient resilience so that when snapped into place, this resilience creates an outward bias on tab 136 that causes edge 135 on the tab to overlie the upper edge of the band. The other end of controller 104 is urged against a lower portion of wall 134 to retain the controller in place in the docking station, much as described with respect to the mounting of controller 8 on controller attachment section 6. In another embodiment, as shown in FIG. 17, controller 104 may be placed in an inclined position in a cradle 131 in a docking station 133. When it is desired to again use RIC system 100, the controller 104 may be removed from docking station 130 or 133 and attached to a new cuff 102 to perform a remote ischemic conditioning procedure on another patient.

In another embodiment of the invention, the user interface 141 on the outer face of controller 104 may be substantially identical or similar to that shown with respect to controller 8. Other user interfaces may also be employed with controller 104. User interface 141 may be substantially identical to user interface 140 on control panel 128, as shown in FIG. 16, or user interfaces 140 and 141 may be different. The provision of similar user interfaces on control panel 128 and controller 104 obviates the need to disrupt the sterile field around the patient to get access to the controller 104 to modify, monitor or terminate the RIC procedure. As shown in FIG. 16, in one embodiment, the user interface on both controller 104 and control panel 128 of docking station 130 or 133 may include a start button having an arrow or the word “start” 142, a stop button 144 and a status indicator 146, such as illuminated numbers indicating the cycle number and an illuminated ring 148 indicating the status of each cycle. In either event, controller 104 may be controlled using interface 141 on controller 104, or by using interface 140 on docking station 130 or 133 of control panel 128 in substantially the same fashion.

Another embodiment of this aspect is a method of use of RIC system 100. RIC system 100 may be used during surgery, during angioplasty, during the implantation of stents or during angiography to reduce or prevent restenosis of a stent being implanted, to reduce any ischemia/reperfusion injury resulting from cessation of blood flow during a procedure, or to reduce traumatic injury. RIC system 100, in suitable cases, may also be used to treat a patient who may suffer from a myocardial infarction, who is at the present time suffering from a myocardial infarction, or who has in the past suffered from a myocardial infarction.

In use, inflatable cuff 102 is wrapped around a limb, such as an upper arm of a patient, typically prior to the start of any medical procedure. Controller 104 is removed from docking station 130 or 133 and is attached to cuff 102 on the patient. Alternatively, controller 104 may be attached to cuff 102 before cuff 102 is wrapped about a limb. A cycle of RIC may be initiated at a desired time before, during and/or after a medical procedure. Typically, the RIC cycle is initiated at user interface 140, although it could be initiated at user interface 141. Controller 104 may be pre-programmed for a predetermined number of cycles, or an operator could manually control the cycles of RIC from control panel 128 or both. Typically, diastolic and systolic blood pressure measurements are obtained between cycles of RIC and are transmitted to controller 127. The cycles of RIC may be discontinued at any time by pressing the stop button 144, by not again pressing start button 142 or by allowing the pre-programmed RIC regimen to run its course. Once the medical procedure has been completed, controller 104 may be removed from cuff 102 and returned to docking station 130 or 133 and cuff 102 may be removed from the patient and discarded or sterilized for future use. Data received from controller 104 may be sent to another designated location for processing and use.

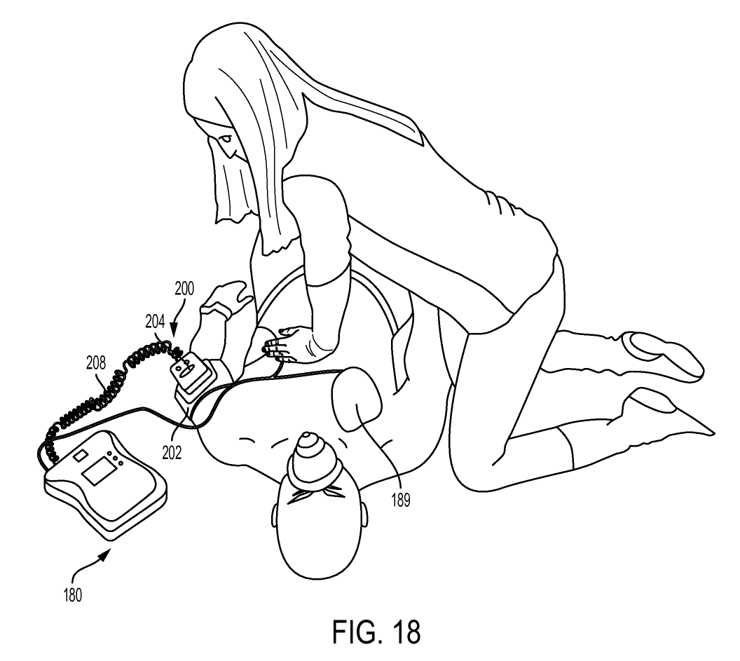

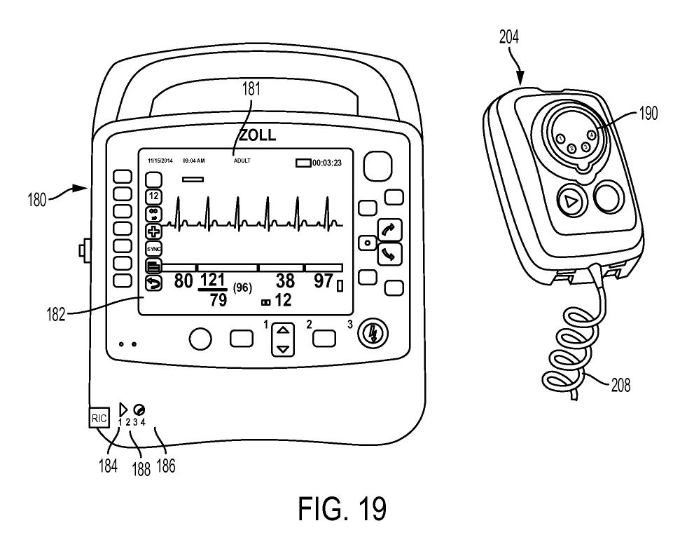

In yet another embodiment of the aspect of the invention in which the RIC system of FIGS. 1-11 may be integrated into other medical apparatus, FIGS. 18 and 19 show the integration of RIC system 200 into a device 180, which may be an automated external defibrillator (AED) and/or an AED/monitor. Device 180 may be any existing device. Examples include, but are not limited to: HeartStart MRx and HeartStart AED, both of which are sold by Philips; LifePak 12 and LifePak CRPlus AED, both of which are sold by Physio-Control International, Inc.; and Zoll AED Plus and Zoll Series X monitor defibrillator, both of which are sold by Zoll Medical Corporation. Device 180 includes one or more paddles or pads 189. Like RIC system 100, RIC system 200 includes an inflatable cuff 202 and a controller 204. Controller 204 may be removably attached to inflatable cuff 202 in the same manner as discussed above with respect to FIGS. 1 and 2, using a controller attachment section (not shown) which may be the same as attachment section 6. Alternatively, controller 204 may be fixedly attached to inflatable cuff 202. Inflatable cuff 202 may have substantially the same configuration and construction as discussed above with respect to inflatable cuff 4. Controller 204 may be substantially the same as controller 8, in one embodiment. In yet another embodiment, controller 204 may be substantially the same as controller 104 in that controller 204 does not include any batteries but includes a pump (not shown) similar to pump 62. RIC system 100 may be used to perform a RIC procedure and to take diastolic and systolic blood pressure measurements between cycles of RIC and these measurements are provided to controller 204. User interface 190 of controller 204 may be substantially the same as user interface 141 on controller 104. Including the pump in controller 204 obviates the need for pneumatic tubing to extend from device 180 which could interfere with the functionality of device 180. This configuration also minimizes any regulatory issues, avoids any unnecessary increase in size or weight of device 180 and permits the RIC system to be more easily upgraded. Power to controller 204 may be provided by a link such as cable 208 which is connected to the power source for AED device 180. As with controller 104, typically a direct current voltage of about 5-12 volts is provided. Cable 208 may also include a data connection between device 180 and controller 204 to allow control of controller 204, as well as monitoring of the progress of the RIC procedure, on the control panel 182 of device 180, and transmission of blood pressure measurements to device 180. The link between device 180 and controller 204 may also be wireless. As illustrated in FIG. 19, an RIC icon may be provided on control panel 182 along with start and stop icons 184 and 186 which permit stopping and starting of the operation of controller 204. Moreover, as shown, numbers 188, such as 1, 2, 3 and 4 may be provided on control panel 182 to indicate the number of RIC procedures, and/or the number of cycles which have been performed as well as the status of the treatment. Icons 184 and 186 may be either firm or soft buttons. As in RIC system 100, these features on user interface 181 on control panel 182 may duplicate the features found on user interface 190 of controller 204 to permit control of the RIC system either on user interface 181 of control panel 182 of device 180, or on user interface 190 of controller 204. This provision of dual control by user interfaces 181 and 190 eliminates the need to step or reach over the patent to start, stop or monitor the status of RIC system 200. Data from controller 204, such as the number of RIC procedures performed, diastolic and systolic blood pressure measurements and other measurements may be provided to device 180 through cable 208 or wirelessly for storage in the controller of device 180. In this manner, this data, along with other data normally collected by device 180 may be stored and transmitted to a hospital or heart center. For example, cuff 202 may be sp-10 compliant to allow for measurement of blood pressure between treatment cycles and transmission of the data to device 180 for later transmission to a hospital or heart center.

A method of use of device 180 and RIC system 200 will now be described. Usually an emergency condition exists when device 180 is used with a patient. The patient may be in cardiac arrest. Thus, RIC system 200 normally is pre-programmed to perform a RIC regimen specific to this type of emergency. For example, controller 204 may be programmed to perform 4 cycles of RIC in which the occlusion and reperfusion durations are about 5 minutes each. However, other occlusion and reperfusion durations and cycle numbers may be used. An emergency responder will affix cuff 202 with or without controller 204 to a patient's upper arm either before, during or after use of the paddles or pads 189. Typically, the cuff will be applied before application of a charge by paddles or pads 189. If cuff 202 is wrapped about an arm without controller 204, controller 204 is then attached to cuff 202. A RIC regimen is initiated by pressing the start icon 184 on control panel 182. Once icon 184 is pressed, a RIC regimen will be performed automatically without need of further action by the first responder. Thereafter, the regimen will stop automatically, or the first responder can stop it at an earlier time by pressing the stop icon 186. If the first responder wants to repeat the regimen, the start icon 184 may be pressed again. Diastolic and systolic blood pressure may be measured after each cycle and provided to device 180. At a time before, during or after performance of the RIC regimen, the first responder may press paddles or pads 189 against the patient's chest and apply a shock in accordance with the normal operation of device 180. When the procedure is completed, the first responder may remove controller 204 from cuff 202 and dispose of cuff 202. Controller 204 is removed with device 180. Data regarding blood pressure and other data sent from controller 204 to device 180 may be stored in device 180 and/or sent to a hospital or heart center.

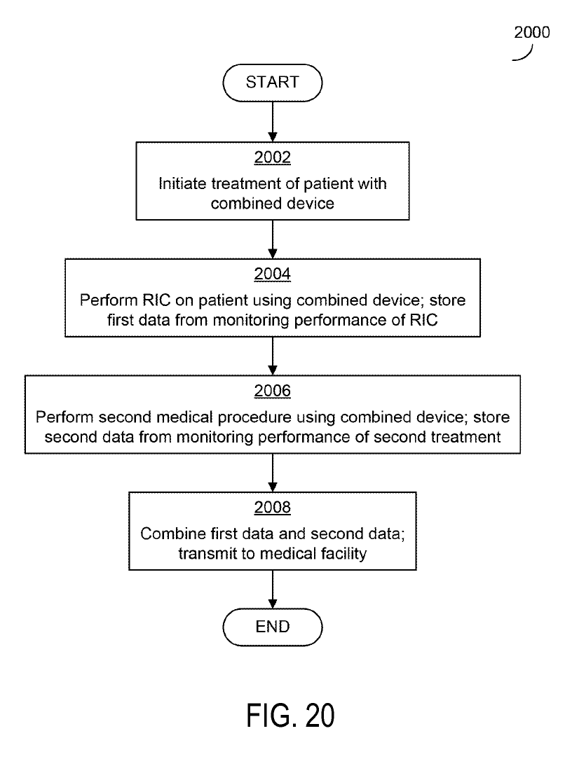

FIGS. 20-23 illustrate examples of processes that may be implemented by a combined medical device to operate the combined medical device to perform RIC on a patient as well as to perform at least one other medical procedure on the patient. As should be appreciated from the foregoing, the other medical procedure may be, for example, monitoring a blood pressure, monitoring a heart rate/rhythm of the patient, monitoring performance of chest compressions, or any of various other examples of medical procedures discussed above. Accordingly, in some embodiments, the combined medical device may be adapted to operate as a RIC device as well as to operate as a sphygmomanometer (blood pressure monitor), an electrocardiogram, a chest compression monitor, or any of various other devices.

FIG. 20 is a flowchart of a process that may be implemented by a combined medical device to transmit data generated by the device to a medical facility. The medical facility may be a facility that will treat the patient, such as in a case where the combined medical device is disposed inside or integrated with an emergency vehicle (e.g., ambulance) that is transporting a patient to a hospital and the combined medical device transmits the data to the hospital. As another example, the medical facility may be a facility that stores medical information, including medical information for the patient on which the combined medical device is to operate. Such a medical facility may be an office of a doctor who has treated the patient, such as a primary care doctor of the patient, or a facility associated with the office of a doctor who has treated the patient, such as a facility that stores medical information on behalf of the doctor's office.

In some embodiments, the combined medical device may include one or more wireless transceivers to transmit the data generated by the device and/or receive data at the device. For example, the combined medical device may include a wireless wide-area network (WWAN) transceiver, such as a cellular transceiver, to transmit the data. In other embodiments, the combined medical device may be communicatively linked to another component that includes a wireless transceiver. For example, in embodiments in which the combined medical device is disposed in or integrated with an emergency vehicle or operated in a patient's home, a wireless transceiver may be disposed in or integrated with the emergency vehicle, and the combined medical device may be communicatively linked to the wireless transceiver. The combined medical device may, for example, be connected to a same computer communication network or other wired and/or wireless link as the wireless transceiver, such as an Ethernet network, an IEEE 802.11 network, a Bluetooth (including Bluetooth Low Energy) network, or other link. Such a wireless transceiver may, in some embodiments, be a mobile device, such as a mobile phone or tablet computer or other device that is operated by a technician of the emergency vehicle, or another user.

The process 2000 of FIG. 20 begins in block 2002, in which the combined medical device initiates treatment of a patient. The combined medical device may initiate treatment in response to receiving input via a user interface, which may be a user interface by which input is provided to both initiate RIC and to initiate performance of the other medical procedure(s), such as examples of user interfaces described above.

When treatment is initiated, the combined medical device will perform both RIC and at least one other medical procedure. The procedures may be performed concurrently and/or may be performed at different times in succession, as discussed above. In some embodiments in which the procedures are performed in succession, the two procedures may alternate, while in other embodiments the procedures may be performed with different frequencies, such that one procedure may be performed multiple times before another procedure is performed. In some embodiments, for example, the combined medical device may perform one of the other procedure(s) multiple times before performing RIC. As a particular example, as discussed in detail below, the combined medical device may perform RIC in response to some condition being satisfied during performance of the other procedure(s).

However the procedure(s) are performed, the combined medical device performs RIC and performs one or more other procedures. In block 2004, during performance of RIC, the combined medical device stores first data that results from monitoring performance of RIC. The combined medical device may generate all of the first data in some embodiments, or may receive some or all of the first data from one or more other sensors during performance of RIC. The first data may relate to the performance of RIC on the patient by the combined medical device. For example, the first data may relate to the manner in which the combined medical device is configured to perform RIC, such as relating to a pressurization of an inflatable cuff during RIC, a length of an ischemic and/or reperfusion period, a number of cycles completed, time at which RIC was performed, or other information relating to operation of the device. As another example, the first data may additionally or alternatively relate to the patient during performance of RIC, such as biological characteristics of the patient detected during RIC. For example, a heart rate, blood pressure, temperature, or other information regarding the patient may be sensed during performance of RIC on the patient by the combined medical device. The information regarding the patient may be sensed by sensors, which may be integrated with the combined medical device and/or connected to the combined medical device. For example, the sensor(s) may be integrated into an inflatable cuff, wirelessly connected to the combined medical apparatus, and/or connected to the combined medical apparatus via a wire.

Similarly, in block 2006, the combined medical device performs a second medical procedure and, during the performance, stores second data. The combined medical device may generate all of the second data in some embodiments, or may receive some or all of the second data from one or more other sensors during performance of the second medical procedure. The second data may relate to the performance of the second medical procedure on the patient by the combined medical device, such as by relating to a manner in which the combined medical device is configured to perform the procedure or is operated to perform the procedure, or relating to biological characteristics of the patient during performance of the second medical procedure. The second data may include, for example, an electrocardiogram trace, information on a number or intensity of chest compressions, or other information.

In block 2008, the combined medical device combines the first data and the second data into one or more messages and transmits the message(s) to the medical facility. As discussed above, the device may transmit the message(s) via a wireless transceiver integrated into the device, or via a transceiver to which the device is communicatively linked. Once wirelessly transmitted, the data may be relayed to the medical facility via one or more networks, such as one or more wired and/or wireless networks, including the Internet.

Once the message(s) are transmitted, the process 2000 ends. Following the process 2000, the data regarding performance of RIC and the other procedure on the patient is stored by the medical facility, where it may be made available to medical practitioner(s), such as medical practitioner(s) who may treat the patient soon after the combined medical device performs RIC and the other medical procedure(s). The other medical practitioner(s) may tailor their treatment of the patient based on the received information regarding performance of the procedures.

While FIG. 20 was described in connection with a wireless transmission of data from the combined medical device, it should be appreciated that embodiments are not so limited and that, in some embodiments, the data may be transmitted using a wired connection.

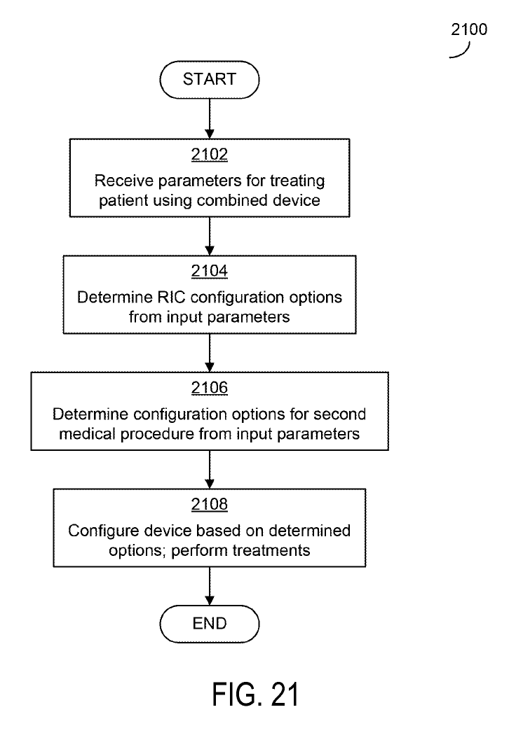

FIG. 21 illustrates another example of a process that may be implemented in some embodiments by a combined medical device that is adapted to perform RIC and one or more other medical procedures. The process 2100 of FIG. 21 may be used by a combined medical device to configure the combined medical device to perform medical procedures on a patient. In particular, through the process 2100 of FIG. 21, a user—who may be a medical practitioner, the patient, or another user—operates a user interface of the combined medical device to select options for treatment and, through the selection of the options, configure the combined medical device to perform both RIC and at least one other medical procedure.

The process 2100 begins in block 2102, in which the combined medical device receives from a user input identifying parameters for treating the patient using the combined medical device. The input may be any suitable input regarding the medical procedures and/or the patient. For example, the user may input information regarding the patient, such as age, height, weight, blood pressure, information about medical history, or other information. As another example, the user may input information regarding a type of treatment to be performed, such as athletic conditioning treatments, prophylactic medical treatments, or treatments for a condition the patient is currently experiencing, and information on how to perform the treatment, such as a length of treatment.

Based on the input received from the user in block 2102, the combined medical device determines configuration options for both RIC and for a second medical procedure. The configuration options may be options other than what are input by the user and received in block 2102, and thus are different from the parameters received in block 2102.

In blocks 2104, the combined medical device determines RIC configuration options from the input parameters, while in block 2106 the combined medical device determines configuration options for the second medical procedure based on the input parameters. For example, if the user inputs in block 2102 that an athletic conditioning treatment is to be performed, in addition to selecting options for a second medical procedure in block 2106, in blocks 2104 and 2106 the combined medical device may select options for performing RIC and for performing a second medical procedure under an athletic conditioning regimen. For example, the combined medical device may select a pressurization, a length of cycle periods, and a number of cycles with which to perform RIC under an athletic conditioning regimen. Similarly, the combined medical device may select options to perform a second medical procedure, such as for monitoring a heart rate or other cardiac properties of a patient under an athletic conditioning regimen, which may alert a user when the user's heart rate or other cardiac properties are in an ideal or beneficial training state or are in an abnormal or dangerous state. As another example, the user may input in block 2102 that the patient is or may be experiencing a heart attack and that treatment for a heart attack is to be given using the combined medical device. In response, in blocks 2104 and 2106, the combined medical device may set options for performing RIC and another medical procedure for a heart attack. For example, the device may set options for performing automated external defibrillation, or other defibrillation, for a heart attack, which may include setting a frequency with which to shock the patient and a voltage to apply. The device may then also set options for performing RIC, including a pressurization, a length of cycle periods, and a number of cycles with which to perform RIC to treat a heart attack.

As another example, if biological information for the patient has been input in block 2102, in blocks 2104 and 2106 the biological information may be used to set configuration options by which the medical procedures are performed. For example, if the biological information indicates a particular blood pressure of the patient, a configuration option for RIC may be set in block 2104 to ensure that the inflatable cuff is pressurized during ischemic periods to at least a pressure that exceeds the patient's blood pressure. Similarly, in block 2106, the patient's blood pressure may be used to set one or more options of a cardiac procedures, such as one or more options for an angiogram or angioplasty.

Once the configuration options are determined in blocks 2104, 2106, the combined medical device configures itself with the determined configuration options, to perform RIC and the second medical procedure in accordance with the determined options, and performs one or both procedures. Once one or both procedures are performed, the process 2100 ends.

The process 2100 illustrated an example of a process that may be used in embodiments to select configuration options on performance of medical procedures based on input from a user. It should be appreciated that configuration options for medical procedures to be performed by a combined medical device may be set in other ways. For example, in some embodiments, data generated during performance of one medical procedure may be used to set configuration options for performance of another medical procedure.

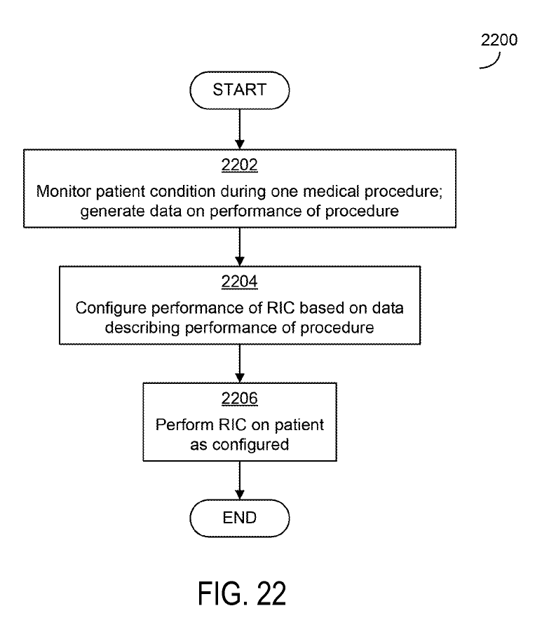

FIG. 22 illustrates an example of a process that may be used in some embodiments to set configuration options for performance of one medical procedure based on data generated during performance of another medical procedure, and/or an evaluation of that data. The process may be implemented by a combined medical device that is adapted to perform both medical procedures. One of the medical procedures may be RIC. In examples described below, the combined medical device determines configuration options for performance of RIC based on data generated during performance of another medical procedure.

The process 2200 of FIG. 22 begins in block 2202, in which a patient's condition is monitored during performance of the other medical procedure and data is generated and stored regarding performance of the procedure on the patient. The data that is generated may include biological characteristics of the patient, detected by one or more sensors. For example, a heart rate, blood pressure, temperature, or other information regarding the patient may be sensed during performance of the other medical procedure on the patient using the combined medical device. The data that is generated may additionally or alternatively relate to a manner in which the second medical procedure is performed on the patient, such as a time at which the procedure is performed, a length of time the procedure is performed, a frequency with which the procedure is performed, or other specific options relating to the type of procedure.

In block 2204, the combined medical device evaluates the data generated in block 2202 and, based on the evaluation, sets configuration options for performance of RIC on the patient. The configuration options that are set include any suitable parameter regarding performance of RIC. For example, a frequency with which RIC treatments are performed, a length of ischemic and/or reperfusion periods during a cycle, a number of cycles in a treatment, a pressurization to maintain in a cuff, whether to start or stop performance of RIC, or other settings may be set based on an evaluation of the data generated during performance of the other medical procedure on the patient.

The evaluation of block 2204 may include various types of evaluation, as embodiments are not limited in this manner. For example, in some embodiments, the combined medical device may compare a value for a biological characteristic of the patient to a threshold to determine whether the value is above or below the threshold, and set a configuration option based on the result. As one specific example, the biological characteristic may be a blood pressure of the patient, which the combined medical device may compare to a threshold to determine whether the patient's blood pressure is above a threshold.

As another example of a type of evaluation that may be performed in some embodiments, the combined medical device may compare a biological characteristic of the patient, such as a value or a series of values of a characteristic, to stored information describing a normal or abnormal state of the biological characteristic. For example, if the biological characteristic is an electrocardiogram trace of the patient, the combined medical device may compare the electrocardiogram trace to stored information indicating how normal and/or abnormal traces appear. As a specific example of comparing an electrocardiogram trace to stored information, the combined medical device may identify a length of an ST segment in the electrocardiogram trace and compare that length of the ST segment to stored information on a normal length and/or an abnormal length. Other information about an electrocardiogram trace, or information on other biological characteristics may be compared to stored information indicating a normal or abnormal state.

As a result of the evaluation in block 2204, the combined medical device sets one or more options for performance of RIC. For example, a pressurization of an inflatable cuff during RIC, a length of an ischemic and/or reperfusion period, a number of cycles to be performed in a treatment, a time at which to perform RIC, a time interval or frequency with which to perform RIC treatments, whether to start performing RIC or stop performing RIC (e.g., start/stop immediately, or start/stop after a time interval), or other information relating to operation of the device to perform RIC on the patient may be set. As a specific example, if the patient's blood pressure is determined to be above a threshold, then the combined medical device may set a pressurization of the inflatable cuff during RIC to be above the patient's blood pressure, while if the patient's blood pressure is determined to be below the threshold, a default pressurization may be used. As another specific example, if the patient's electrocardiogram trace is determined to be abnormal, such as by including a shorted ST segment, then the combined medical device may begin performing RIC if it was not previously performing RIC.

The biological characteristics may also be evaluated over time and the combined medical device may set configuration options for RIC based on how one or more biological characteristics of the patient change over time. For example, if the evaluation of the biological characteristic(s) shows that the biological characteristic(s) have changed over time from a normal state to an abnormal state, or vice versa, or changed from being above a threshold to below a threshold, or vice versa, then the combined medical device may respond by changing one or more configuration options for performing RIC. As a specific example, if before the change the combined medical device was not performing RIC or performing RIC at one time interval or frequency, the combined medical device may start performing RIC or performing RIC at another interval/frequency. Similarly, if before the change the combined medical device was performing RIC, the combined medical device may stop performing RIC.

In some embodiments, the combined medical device may automatically set the configuration option(s) determined through the evaluation of block 2204. In other embodiments, the combined medical device may prompt a user—such as the patient, a medical practitioner, or other user—with recommendations for changes to configuration option(s) and may not change the configuration option(s) until receiving verification from the user to make the change.

In block 2206, once the configuration options are set, the combined medical device performs RIC in accordance with the configuration options.

In some embodiments, a combined medical device may be adapted to perform RIC, but performance of RIC using the combined medical device may require changes to be made to the device following performance of RIC. For example, performance of RIC may require that one or more components of the combined medical device be replaced.

An inflatable cuff of a combined medical device may be used in a number of different procedures, as should be appreciated from the foregoing. The inflatable cuff may be used, for example, as part of a sphygmomanometer to monitor a blood pressure of a patient and may be used to perform RIC. Performing RIC may involve pressurizing the inflatable cuff to a pressurization higher than a pressurization reached when the inflatable cuff is pressurized during monitoring of a patient's blood pressure. There may be concern that pressurization of the inflatable cuff reaches a degree that the inflatable cuff may be deformed or may lose some structural integrity, and thus it may be advisable not to pressurize an inflatable cuff once it has been used for RIC.

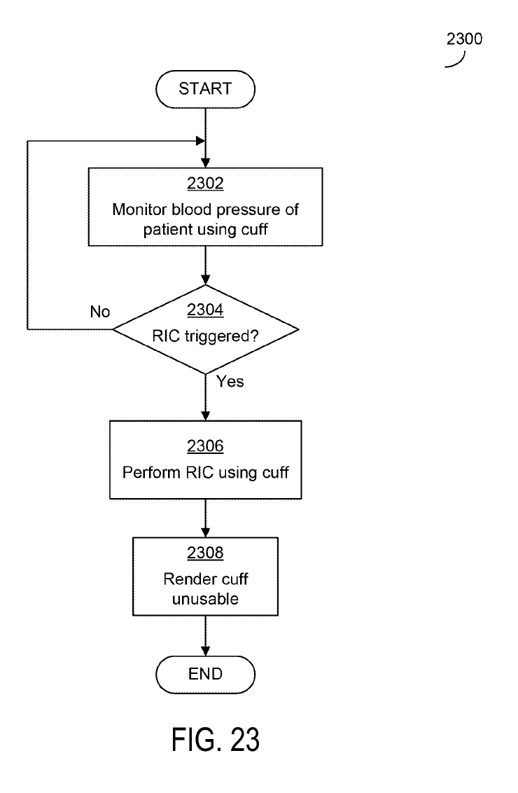

Accordingly, in some embodiments, a combined medical device may monitor usage of components of a combined medical device to determine whether one or more components of the combined medical device should be replaced following specific usage of the components, including usage of the components to perform RIC. FIG. 23 illustrates an example of a process that may be used in some embodiments to determine whether one or more components of a combined medical device, which may be used in both RIC and in one or more other medical procedures, should be replaced. Examples below will be described in connection with detecting usage of an inflatable cuff, but other components of the combined medical device may be used.

The process 2300 of FIG. 23 begins in block 2302, in which a combined medical device operates an inflatable cuff of the device any number of times to monitor the blood pressure of a patient or of more than one patient. As shown in the loop of blocks 2302, 2304 of FIG. 23, the combined medical device may perform the monitoring of block 2302 multiple times. In block 2304, the combined medical device determines whether the combined medical device has been triggered to perform RIC on a patient. The combined medical device may be triggered through direct user input, or through evaluating one or more biological characteristics and starting performance of RIC in response to the evaluation, as discussed above. If the combined medical device determines that performance of RIC has been triggered, then in block 2306 the combined medical device operates the inflatable cuff in accordance with a RIC treatment protocol. In addition, following performance of RIC in block 2306, in block 2308 the combined medical device renders the inflatable cuff inoperable.

The device may render the cuff inoperable in various ways, as embodiments are not limited in this respect. For example, in some embodiments, the combined medical device may include a storage medium, such as a computer memory, that indicates whether the inflatable cuff has been used in RIC. In such embodiments, prior to operation of the inflatable cuff, the combined medical device may retrieve data from the storage medium to determine whether the data indicates that the cuff has previously been used in RIC. If the storage medium stores data indicates that the inflatable cuff has been used in RIC, then the combined medical device may not pressurize the inflatable cuff and may output a message to a user that RIC cannot be performed with the inflatable cuff.

In some embodiments that include such a storage medium, the storage medium may be included in a controller of the combined medical device. In some embodiments in which the controller and the inflatable cuff are removably coupled to one another, and in which the controller may be used with multiple different inflatable cuffs, the storage medium may be integrated with the inflatable cuff. In some such embodiments, after performing RIC using the inflatable cuff, the controller may write data to the storage medium of the inflatable cuff indicating that RIC was performed. After the data is stored, a user would replace the used cuff with a new inflatable cuff before the combined medical device could be used to perform RIC again.

Once the inflatable cuff is rendered inoperable in block 2308, the process 2300 ends.

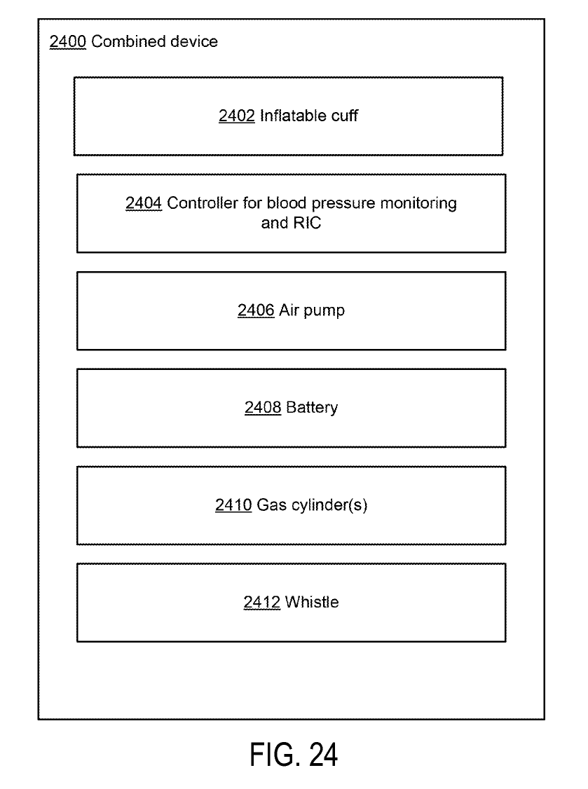

Various examples of combined medical devices, and processes for operating combined medical devices, are described above. FIG. 24 illustrates, in the form of a block diagram, another example of a combined medical device. The device 2400 of FIG. 24 is adapted to both monitor blood pressure as a sphygmomanometer and perform RIC. FIG. 24 illustrates some examples of components of such a combined device.

The device 2400 includes both an inflatable cuff 2402 and a controller 2404 to operate the cuff 2402 as to monitor blood pressure and to perform RIC. The cuff 2402 and controller 2404 may be implemented in accordance with examples described above, or in other ways. To inflate the inflatable cuff 2402 during monitoring of blood pressure, the controller 2404 may operate an air pump 2406 using electric power provided by a battery 2408. The battery 2408 may provide sufficient power to drive the air pump 2406 to pressurize the inflatable cuff 2402 a number of times to monitor blood pressure. However, pressurizing the inflatable cuff 2404 to perform RIC requires increasing the pressure in the cuff 2402 to a degree higher than during monitoring of blood pressure, and thus requires driving the air pump 2406 for a longer period. Driving the air pump 2406 with the battery 2408 to inflate the cuff 2402 for RIC may therefore drain the battery 2408, which may be undesirable. In addition, the combined medical device 2400 may be used to perform RIC in emergency situations in some cases, such as where RIC is performed when a patient may be experiencing a heart attack. Relying on battery 2408 to drive the air pump 2406 may therefore be dangerous, as there is a risk that there may not be sufficient power in the battery 2408 to drive the air pump 2406 to properly perform RIC using the device 2400.

Accordingly, the device 2400 includes one or more pressurized gas cylinders 2410. The device 2400 may be arranged such that when a seal of one or more of the cylinders 2410 is broken, the pressurized gas from the cylinder flows into the cuff 2402 and pressurizes the cuff 2402. Breaking the seal of the cylinder 2410 may return much less power than driving the air pump 2406 and thus requires less power to be drawn from the battery 2408. In some embodiments, including the example of FIG. 24, the device 2400 may also include a whistle 2412, which is arranged in the device 2400 such that pressurized air flowing out of the cylinder(s) 2410 flows into the whistler 2412 and causes the whistle 2412 to emit a sound. The sound may be useful where the device 2400 is being used in an emergency situation, as the sound may alert others that the device 2400 is being used to perform RIC and alert them to the emergency.

Techniques operating according to the principles described herein may be implemented in any suitable manner. Included in the discussion above are a series of flow charts showing the steps and acts of various processes that may be implemented by a combined medical device that is adapted to perform RIC and a second medical procedure. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application. It should be appreciated that embodiments are not limited to being implemented in any specific number, division, or type of functional facilities. In some implementations, all functionality may be implemented in a single functional facility, while in other embodiments multiple functional facilities may be used.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as a portion of a computing device or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

What is claimed is:

1. A method of configuring performance of remote ischemic conditioning (RIC) on a patient that is being treated using a medical apparatus that is adapted to perform RIC and at least one second medical procedure, the method comprising:
operating at least one processor of the medical apparatus to carry out acts of:
monitoring one or more biological characteristics of the patient during performance of one or more of the at least one second medical procedure on the patient using the medical apparatus;
evaluating the one or more biological characteristics to identify a manner in which to perform RIC on the patient using the medical apparatus; and
configuring the medical apparatus to perform RIC on the patient in the manner identified as a result of the evaluating.

2. The method of claim 1, wherein:
monitoring the one or more biological characteristics comprises monitoring blood pressure of the patient;
evaluating the one or more biological characteristics comprises determining whether the blood pressure of the patient is above a threshold; and
configuring the medical apparatus comprises, in response to determining that the blood pressure of the patient is above the threshold, increasing a pressure to which a cuff of the medical device is to be inflated during an ischemic period of the RIC.

3. The method of claim 1, wherein evaluating the one or more biological characteristics comprises determining whether the one or more biological characteristics change between a normal state and an abnormal state during performance of the one or more of the at least one second medical procedure.

4. The method of claim 3, wherein configuring the medical apparatus comprises, in response to determining that the one or more biological characteristics have changed between the normal state and the abnormal state, initiating performance of RIC on the patient using the medical apparatus.

5. The method of claim 3, wherein:
the method further comprises operating the at least one processor to carry out an act of, during performance of the one or more of the at least one second medical procedure on the patient using the medical apparatus, intermittently performing RIC on the patient using the medical apparatus; and
configuring the medical apparatus comprises, in response to determining that the one or more biological characteristics have changed between the normal state and the abnormal state, ceasing performance of RIC on the patient using the medical apparatus.

6. The method of claim 3, wherein:
the method further comprises operating the at least one processor to carry out an act of, during performance of the one or more of the at least one second medical procedure on the patient using the medical apparatus, intermittently performing RIC on the patient using the medical apparatus; and
configuring the medical apparatus comprises, in response to determining that the one or more biological characteristics have changed between the normal state and the abnormal state, adjusting a frequency with which RIC is performed on the patient during performance of the one or more of the at least one second medical procedure.

7. The method of claim 3, wherein:
the method further comprises operating the at least one processor to carry out an act of, during performance of the one or more of the at least one second medical procedure on the patient using the medical apparatus, intermittently performing RIC on the patient using the medical apparatus; and
configuring the medical apparatus comprises, in response to determining that the one or more biological characteristics have changed between the normal state and the abnormal state, prompting a user of the medical apparatus for whether to adjust a frequency with which RIC is performed on the patient during performance of the one or more of the at least one second medical procedure.

8. The method of claim 3, wherein determining whether the one or more biological characteristics change between the normal state and the abnormal state comprises determining whether the one or more biological characteristics change from the normal state to the abnormal state.

9. The method of claim 3, wherein:
the one or more biological characteristics comprises a first biological characteristic; and
determining whether the one or more biological characteristics change between the normal state and the abnormal state comprises comparing information on the first biological characteristic for the patient to stored information on a normal state and/or an abnormal state of the first biological characteristic.

10. The method of claim 9, wherein:
the first biological characteristic is a characteristic of an electrocardiogram trace of the patient; and
determining whether the one or more biological characteristics change between the normal state and the abnormal state comprises comparing the characteristic of the electrocardiogram for the patient to stored information on a normal electrocardiogram; and
configuring the medical apparatus to perform RIC on the patient in the manner identified as a result of the evaluating comprises initiating performance of RIC using the medical apparatus in response to determining, as a result of the comparing, that the electrocardiogram trace of the patient is abnormal.

11. The method of claim 9, wherein:
the first biological characteristic is length of an ST segment on an electrocardiogram trace of the patient; and
determining whether the one or more biological characteristics change between the normal state and the abnormal state comprises comparing a length of an ST segment for the patient to stored information on a normal length of an ST segment; and
configuring the medical apparatus to perform RIC on the patient in the manner identified as a result of the evaluating comprises initiating performance of RIC using the medical apparatus in response to determining, as a result of the comparing, that the patient is showing ST elevation.

12. The method of claim 1, wherein evaluating the one or more biological characteristics to identify a manner in which to perform RIC on the patient using the medical apparatus comprises determining, based on the one or more biological characteristics, one or more configuration options from a group of configuration options consisting of pressure to which to inflate a cuff during an ischemic period, a length of an ischemic period, a length of a reperfusion period, and a number of cycles.

13. At least one non-transitory computer-readable storage medium having encoded thereon executable instructions that, when executed by at least one processor of a medical apparatus adapted to perform remote ischemic conditioning (RIC) and at least one second medical procedure, cause the at least one processor to carry out a method of configuring the medical apparatus to perform RIC on a patient, the method comprising:
monitoring one or more biological characteristics of the patient during performance of one or more of the at least one second medical procedure on the patient using the medical apparatus;
evaluating the one or more biological characteristics to identify a manner in which to perform RIC on the patient using the medical apparatus; and
configuring the medical apparatus to perform RIC on the patient in the manner identified as a result of the evaluating.

14. A method of operating a medical apparatus that is adapted to perform remote ischemic conditioning (RIC) and at least one second medical procedure on the patient, wherein the medical apparatus is disposed within an emergency vehicle transporting the patient to a medical facility, the method comprising:
generating, during performance of RIC on the patient using the medical apparatus, first data regarding the performance of the RIC on the patient;
generating, during performance of the at least one second medical procedure on the patient, second data regarding the performance of the at least one second medical procedure on the patient;
triggering wireless transmission of at least one message comprising the first data and the second data to the medical facility.

15. An apparatus comprising:
an inflatable cuff; and
a controller to control inflation of the inflatable cuff in accordance with a protocol, wherein the controller comprises:
at least one processor; and
at least one storage medium having encoded thereon executable instructions that, when executed by the at least one processor, cause the at least one processor to carry out a method comprising:
in response to user input requesting operation of the apparatus as a blood pressure monitor, controlling inflation of the inflatable cuff in accordance with a first protocol associated with blood pressure monitoring; and
in response to user input requesting operation of the apparatus to perform remote ischemic conditioning (RIC) on a patient,
controlling inflation of the inflatable cuff in accordance with a second protocol associated with RIC, and
rendering the inflatable cuff inoperable once RIC has been performed using the inflatable cuff.

16. The apparatus of claim 15, wherein the inflatable cuff and the controller are removably coupled.

17. The apparatus of claim 15, wherein the method further comprises:
following inflation of the inflatable cuff in accordance with the second protocol associated with RIC and in response to user input requesting operation of the apparatus as the blood pressure monitor, refraining from inflating the inflatable cuff in accordance with the first protocol.

18. The apparatus of claim 17, wherein the method further comprises:
following inflation of the inflatable cuff in accordance with the second protocol associated with RIC and in response to user input requesting operation of the apparatus as the blood pressure monitor, outputting an error message via a user interface of the controller.

19. The apparatus of claim 15, wherein the method further comprises:
in response to the user input requesting operation of the apparatus as the blood pressure monitor and prior to controlling inflation of the inflatable cuff in accordance with the first protocol, determining whether the inflatable cuff to which the controller is coupled has been rendered inoperable; and
controlling inflation of the inflatable cuff in accordance with the first protocol comprises controlling the inflation in response to determining that the inflatable cuff to which the controller is coupled has been rendered inoperable.

20. At least one non-transitory computer-readable storage medium having encoded thereon executable instructions that, when executed by at least one processor, cause the at least one processor to carry out a method of controlling inflation of an inflatable cuff of a medical apparatus in accordance with a protocol, the method comprising:
in response to user input requesting operation of the medical apparatus as a blood pressure monitor, controlling inflation of the inflatable cuff in accordance with a first protocol associated with blood pressure monitoring; and
in response to user input requesting operation of the medical apparatus to perform remote ischemic conditioning (RIC) on a patient,
controlling inflation of the inflatable cuff in accordance with a second protocol associated with RIC, and
rendering the inflatable cuff inoperable once RIC has been performed using the inflatable cuff.

21. An apparatus comprising:
one or more pressurized gas cylinders;
an air pump;
an inflatable cuff;
a battery;
at least one processor; and
at least one computer-readable storage medium having encoded thereon executable instructions that, when executed by the at least one processor, cause the at least one processor to carry out a method comprising:
in response to user input requesting operation of the apparatus as a blood pressure monitor, operating the air pump to inflate the inflatable cuff and sensing a blood pressure of a patient to which the inflatable cuff is attached; and
in response to user input requesting performance of remote ischemic conditioning (RIC) on a patient using the apparatus, triggering inflation of the inflatable cuff using a pressurized gas cylinder of the one or more pressurized gas cylinders.

22. The apparatus of claim 21, further comprising:
a whistle arranged in the apparatus such that gas released from any one of the pressurized gas cylinders enters the whistle and causes the whistle to emit sound.

23. The apparatus of claim 21, further comprising:
a housing, wherein the one or more pressurized gas cylinders, the air pump, the battery, the at least one processor, and the at least one storage medium are disposed in and/or on the housing,
wherein the inflatable cuff is removably coupled to the housing.

24. Medical apparatus comprising:
a system for performing remote ischemic conditioning, the system comprising:
an inflatable cuff configured to encircle a limb of a patient and to occlude blood flow through the limb of the patient;
a controller attached to the cuff and configured to perform a remote ischemic conditioning procedure on a patient; and
a pump for providing gas to inflate the inflatable cuff, the pump being controlled by the controller;
apparatus for performing a medical procedure on a patient; and
a data link between the system for performing remote ischemic conditioning and the apparatus for performing a medical procedure.

25. The medical apparatus of claim 24, wherein:
the system further comprises a first control panel on a face of the controller;
the apparatus comprises a control panel for controlling operation of the apparatus; and
the system for performing remote ischemic conditioning may be controlled either by the control panel on the face of the controller or by the control panel of the apparatus for performing a medical procedure.

26. A table comprising:
apparatus for performing a medical procedure;
a table surface upon which the medical procedure may be performed;
a system for performing remote ischemic conditioning on a patient before, during and/or after performance of the medical procedure, the system for performing remote ischemic conditioning comprising:
an inflatable cuff configured to encircle a limb of a patient, and to occlude blood flow through the limb of the patient;
a controller attached to the cuff, the controller being configured to alternately inflate and deflate the cuff to alternately occlude blood flow and to allow blood to flow through the limb during a programmed cycle of remote ischemic conditioning; and
a pump for inflating the cuff under the control of the controller; and
a data link between the first control panel and the controller of the remote ischemic conditioning system to allow the transmission of data between the remote ischemic conditioning system controller and the first control panel.

27. The table of claim 26, wherein:
the system further comprises a first control panel on a face of the controller for controlling operation of the controller and monitoring progress of the programmed cycle of remote ischemic conditioning;
the apparatus comprises a second control panel for controlling operation of the apparatus; and
the system for performing remote ischemic conditioning may be controlled either by the control panel on the face of the controller or by the control panel of the apparatus for performing a medical procedure.

28. The table of claim 26, wherein the data link is a wireless data link.

29. An automated external defibrillator comprising:
paddles or pads for applying a charge to a patient;
a first controller for controlling operation of the paddles or pads in accordance with automated external defibrillation;
a system for performing remote ischemic conditioning, the system comprising:
a cuff configured to encircle a limb of a patient;
a pump for providing a gas to the cuff for inflation of the cuff; and
a second controller for controlling the pump to inflate and deflate the cuff to perform cycles of remote ischemic conditioning to the patient, each cycle including a period of occlusion of blood flow through the limb followed by a period of reperfusion of blood flow as the cuff is deflated; and
a data link between the control panel on the first controller and the second controller to provide power to the second controller and to permit data transmission between the second controller and the first controller.

30. The automated external defibrillator of claim 29, wherein:
the system further comprises a user interface on the second controller for controlling operation of the second controller and monitoring progress of remote ischemic conditioning;
the first controller comprises a control panel for controlling operation of the pads or paddles in accordance with automated external defibrillation; and
the system for performing remote ischemic conditioning may be controlled either by the control panel on the face of the controller or by the control panel of the apparatus for performing a medical procedure.

31. The automated external defibrillator of claim 29, wherein data generated by the second controller is sent to and stored by a memory of the first controller.
